(19) United States
(12) Patent Application Publication
(10) Pub. No.: US 2026/0242861 A1
(43) Pub. Date: Aug. 20, 2026

(54) STATE IDENTIFICATION METHOD

(71) Applicant: Yokogawa Electric Corporation, Tokyo (JP)

(72) Inventors: Tomoyuki TAGUCHI, Tokyo (JP); Yuko HIRAKAWA, Tokyo (JP)

(73) Assignee: Yokogawa Electric Corporation, Tokyo (JP)

(21) Appl. No.: 19/161,360

(22) PCT Filed: Feb. 29, 2024
(86) PCT No.: PCT/JP2024/007566

§ 371 Date: Sep. 2, 2025

(30) Foreign Application Priority Data
Mar. 7, 2023 (JP) 2023-034703

(51) Int. Cl. C12Q 1/686; C12Q 1/6806; G01N 21/29; G01N 21/31

(52) U.S. Cl. CPC C12Q 1/686; C12Q 1/6806; G01N 21/29; G01N 21/31; G01N 2021/3125

(57) ABSTRACT

A state identification method for identifying the temperature at which a sample has been heated, includes adding a predetermined dye to a sample solution including the sample, heating the sample solution to which the predetermined dye has been added, and identifying the temperature at which the sample solution has been heated on the color tone change of the predetermined dye.

### TECHNICAL FIELD

The present invention relates to a state identification method.

### BACKGROUND ART

There is known a technique of collecting a bacterial culture solution, adding a solubilizing agent, and extracting nucleic acids from the cells of bacteria contained in the bacterial culture solution (referred to as “high-temperature and high-pressure method” as appropriate). In addition, there is known a technique of identifying the temperature at which a container is heated (referred to as “heating temperature” and “heating” as appropriate) by confirming the color tone of a dye such as Prussian blue or a leuco dye that change color according to a temperature in a process requiring a heating treatment of the container.

### CITATION LIST

### Patent Literature

### [PTL 1]

Japanese Patent No. 5624487

### [PTL 2]

Japanese Laid-open Patent Publication 2013-132298

### [PTL 3]

Japanese Laid-open Patent Publication 2002-322385

### SUMMARY OF INVENTION

### Technical Problem

However, in the above technique, it is difficult to effectively identify the heating temperature of the bacterial culture solution stored in the container. For example, in the above-described method for confirming discoloration of Prussian blue, discoloration occurs at around 121° C., which is lower than around 140° C. at which nucleic acids can be extracted from bacteria, and thus it is difficult to confirm whether the temperature has actually reached around 140° C. In addition, in the method of confirming discoloration of the leuco dye, since discoloration occurs at around 70° C., and the hue reversibly changes to the original color at 20° C., it is difficult to confirm whether the temperature has actually reached around 140° C.

The present invention has been made in view of the above, and an object thereof is to effectively identify the heating temperature of a sample.

### Solution to Problem

According to an aspect of the embodiments, a state identification method for identifying the temperature at which a sample has been heated, includes an addition step of adding a predetermined dye to a sample solution comprising the sample, a heating step of heating the sample solution to which the predetermined dye has been added by the addition step, and an identification step of identifying the temperature at which the sample solution has been heated by the heating step based on the color tone change of the predetermined dye.

### Advantageous Effects of Invention

The present invention has an effect of effectively identifying the heating temperature of a sample.

### BRIEF DESCRIPTION OF DRAWINGS

FIG. 1 is a diagram illustrating a configuration example of a heating identification system according to an embodiment.

FIG. 2 is a diagram illustrating an example of a color component of beet red according to the embodiment.

FIG. 3 is a diagram illustrating an example of experimental result 1 of the heating identification experiment according to the embodiment.

FIG. 4 is a diagram illustrating an example of experimental result 2 of the heating identification experiment according to the embodiment.

FIG. 5 is a diagram illustrating an example of experimental result 3 of the heating identification experiment according to the embodiment.

FIG. 6 is a diagram illustrating an example of experimental conditions 1 of the PCR amplicon measurement experiment according to the embodiment.

FIG. 7 is a diagram illustrating an example of experimental conditions 2 of the PCR amplicon measurement experiment according to the embodiment.

FIG. 8 is a diagram illustrating an example of experimental conditions 3 of the PCR amplicon measurement experiment according to the embodiment.

FIG. 9 is a diagram illustrating an example of experimental result 1 of the PCR amplicon measurement experiment according to the embodiment. FIG. 10 is a diagram illustrating an example of experimental result 2 of the PCR amplicon measurement experiment according to the embodiment.

FIG. 11 is a flowchart illustrating an example of the flow of the heating identification step according to the embodiment.

### DESCRIPTION OF EMBODIMENTS

Hereinafter, a state identification method according to an embodiment of the present invention will be described in detail with reference to the drawings.

Note that the present invention is not limited to the embodiments described below.

### First Embodiment

Hereinafter, the configuration of a heating identification system 100 according to the embodiment, details of each step, and the flow of the steps will be sequentially described, and finally, the effects of the embodiment will be described.

### 1. Configuration of Heating Identification System 100

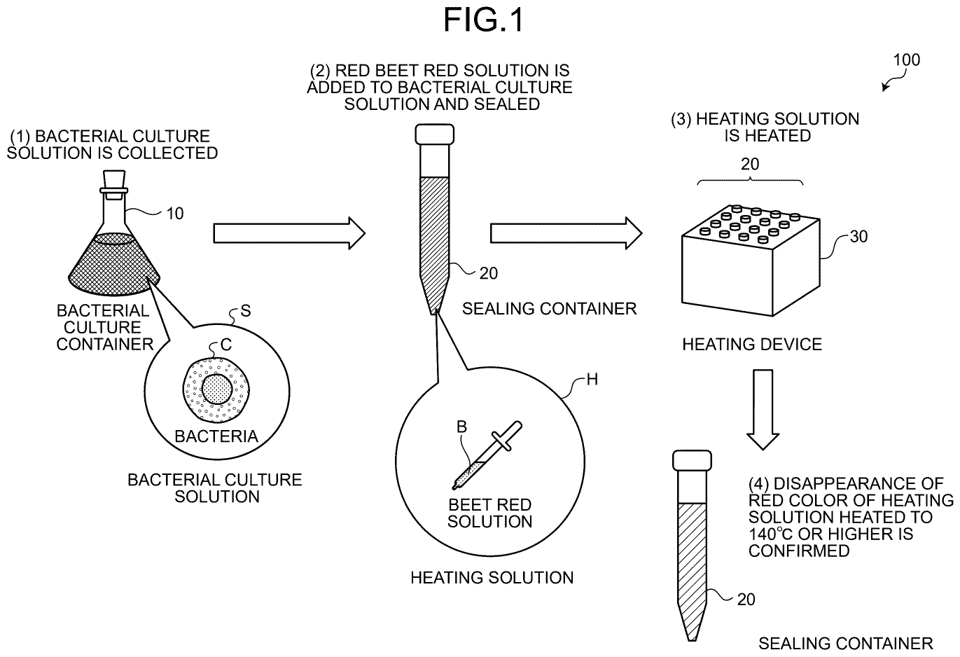

The configuration of the heating identification system 100 according to the embodiment will be described with reference to FIG. 1. FIG. 1 is a diagram illustrating a configuration example of the heating identification system 100 according to the embodiment. Hereinafter, the configuration example of the entire heating identification system 100, examples of the steps of the heating identification system 100, and effects of the heating identification system 100 will be described in this order.

### 1-1. Configuration Example of Entire Heating Identification System 100

The heating identification system 100 includes a bacterial culture container 10, a sealing container 20, and a heating device 30. Hereinafter, the bacterial culture container 10, the sealing container 20, and the heating device 30 will be described in this order.

### 1-1-1. Bacterial Culture Container 10

The bacterial culture container 10 is a container for storing a bacterial culture solution S. The bacterial culture solution S is a solution for culturing a microorganism such as bacteria C. In the example of FIG. 1, the bacterial culture container 10 is a conical flask with a stopper, but the shape, material, capacity, and the like of the bacterial culture container 10 are not limited.

### 1-1-2. Sealing Container 20

The sealing container 20 is a container for sealing a heating solution H. The heating solution H is a solution obtained by adding beet red B, which is a red dye, to the bacterial culture solution S. In the example of FIG. 1, the sealing container 20 is a glass tube with a stopper, but the shape, material, capacity, and the like of the sealing container 20 are not limited.

### 1-1-3. Heating device 30

The heating device 30 is a container that heats the heating solution H. In the example of FIG. 1, the heating device 30 is a heat block, but the shape, material, heating method, and the like of the heating device 30 are not limited.

### 1-1-4. Others

The heating identification system 100 illustrated in FIG. 1 may include a plurality of bacterial culture containers 10, a plurality of sealing containers 20, or a plurality of heating devices 30. Further, the bacterial culture container 10 may be integrated with the sealing container 20.

### 1-2. Example of Steps of Heating Identification System 100

Examples of the steps of the heating identification system 100 will be described. Hereinafter, a bacterial culture solution collection step, a dye addition step, a heating step, and a dye confirmation step will be described in this order. Note that these steps may be executed in a different order. Some of these steps may be omitted.

### 1-2-1. Bacterial Culture Solution Collection Step

First, in the heating identification system 100, the bacterial culture solution collection step illustrated in FIG. 1(1) is performed. For example, in the bacterial culture solution collection step, Escherichia coli(E. coli) or Staphylococcus aureus(S. aureus) is used as the bacteria C, and a part of the bacterial culture solution S cultured overnight at 37° C. in a Soybean Casein Digest (SCD) liquid medium is collected in the sealing container 20 using a sterilized whole pipette.

### 1-2-2. Dye Addition Step

Second, in the heating identification system 100, the dye addition step illustrated in FIG. 1(2) is performed. For example, in the dye addition step, beet red B, which is a red dye, is used, and an aqueous solution of beet red B is added to the sealing container 20 with the bacterial culture solution S collected therein, and the container is sealed by stoppering. At this time, in the dye addition step, a solubilizing agent that promotes the lysis of the cells of the bacteria C may be further added to the sealing container 20.

### 1-2-3. Heating Step

Thirdly, in the heating identification system 100, the heating step illustrated in FIG. 1(3) is performed. For example, in the heating step, the heating device 30 is preheated to a set temperature of 140° C., the sealing container 20 is set in the heating device 30, and the sealing container 20 in which the heating solution H has been sealed is heated at 140° C. for 45 seconds.

### 1-2-4. Dye Confirmation Step

Fourth, in the heating identification system 100, the dye confirmation step illustrated in FIG. 1(4) is performed. For example, in the dye confirmation step, it is visually confirmed that the red color of the beet red B sealed in the sealing container 20 has disappeared, and it is confirmed that the heating solution H in the sealing container 20 has been heated to 140° C. or higher. At this time, in the dye confirmation step, the heating temperature can also be identified according to the decrease in absorbance by measuring the absorbance of the beet red B sealed in the sealing container 20.

### 1-2-5. Others

In the heating identification system 100, the nucleic acid purification step of purifying the nucleic acids of the bacteria C extracted in the heating solution H can be further performed. For example, in the nucleic acid purification step, the nucleic acids extracted from the bacteria C can be purified by injecting the heating solution H after the heating step into a column, and injecting an eluate.

In the heating identification system 100, the nucleic acid amplification step of amplifying the nucleic acids of the bacteria C extracted in the heating solution H can be further performed. For example, in the nucleic acid amplification step, the nucleic acids extracted from the bacteria C can be amplified by adding a polymerase chain reaction (PCR) mix to the heating solution H after the heating step and performing PCR.

### 1-3. Effects of Heating Identification System 100

Hereinafter, overviews of heating identification techniques as reference techniques and improvements required for the reference techniques will be sequentially described, and then effects of the heating identification system 100 will be described.

### 1-3-1. Overview of Reference Technique 1

In the reference technique 1 described in Patent Literature 2, a moisture and heat discoloration indicator composition containing (A) Prussian blue, (B) a gallic acid ester such as propyl gallate, and (C) at least one compound selected from the group consisting of amino acids such as dicyandiamide and sodium glutamate, aromatic carboxylic acids such as benzoic acid, acid amides such as nicotinic acid amide, and saccharides such as starch is made into ink, and the date of manufacture, the best-before expiration, and the like are printed on the surface of a package of a retort pouch food. In the reference technique 1 described above, the moisture and heat discoloration indicator composition exhibits a blue color before the heat sterilization treatment, but exhibits a black color after the treatment, whereby the heat sterilization treatment can be confirmed.

### 1-3-2. Improvements Required for Reference Technique 1

The reference technique 1 requires the following improvements. First, the reference technique 1 confirms discoloration at 121° C., but since the treatment temperature effective for the nucleic acid extraction technique by the high-temperature and high-pressure method described in PTL 1 is 140° C. Therefore, the reference technique 1 in which discoloration occurs at 121° C. cannot confirm whether the temperature has actually reached 140° C. Second, the reference technique 1 requires about 20 minutes for the dye to change in color, but the treatment time for the nucleic acid extraction technique by the high-temperature and high-pressure treatment described in PTL 1 is several tens of seconds. Therefore, there is a high possibility that further treatment will fragment the genomic DNA more than necessary and affect the subsequent nucleic acid amplification step by PCR. From the above, it is difficult to apply the reference technique 1 as a temperature monitoring method in the high-temperature and high-pressure method in which nucleic acids are extracted from the bacteria C.

### 1-3-3. Overview of Reference Technique 2

The reference technique 2 described in PTL 3 provides a temperature-sensitive color-phase reversible composition containing three components: a color former composed of a reversible dye that develops a color with an acid, a color developer composed of a Lewis acid having a melting point in the temperature range of the desired hue change, and a sensitizer, which develops a color when heated and decolors when cooled to exhibit a reversible hue change. In the reference technique 2 described above, examples of suitable color formers include leuco dyes, examples of suitable color developers include long-chain carboxylic acids, and examples of suitable sensitizers include acid amides.

### 1-3-4. Improvements Required for Reference Technique 2

The reference technique 2 requires the following improvements. First, in the reference technique 2, the leuco dye is discolored at 70° C., and the hue is reversibly changed to the original color at 20° C., but since the treatment temperature effective for the nucleic acid extraction technique by the high-temperature and high-pressure method described in PTL 1 is 140° C. Therefore, the reference technique 2 in which discoloration occurs at 70° C. cannot confirm whether the temperature has actually reached 140° C. Second, in the reference technique 2, the leuco dye has a characteristic of developing color in response to an acid, so its hue change may be affected by the properties of the reaction solution, and thus the leuco dye is difficult to use as a process control. From the above, it is difficult to apply the reference technique 2 as a temperature monitoring method in the high-temperature and high-pressure method in which nucleic acids are extracted from the bacteria C.

### 1-3-5. Overview of Heating Identification System 100

In the heating identification system 100, beet red B, which is a red dye, is added to the bacterial culture solution S containing the bacteria C, the bacterial culture solution S with beet red B is heated, and the temperature at which the bacterial culture solution S has been heated is identified based on the color tone change of beet red B. In addition, in the heating identification system 100, nucleic acids are extracted from the cells of the bacteria C by heating. In addition, in the heating identification system 100, the nucleic acids extracted from the cells of the bacteria C are amplified by heating.

### 1-3-6. Effect of Heating Identification System 100

First, the heating identification system 100 can confirm the color tone change at 140° C. That is, since beet red B has a color tone change even at 140° C., the heating identification system 100 can be expected to be applied as a temperature monitoring method in an autoclave, which is a representative sterilization method performed at a temperature higher than 120° C.

Second, in the heating identification system 100, it is easy to confirm the maximum attained temperature. That is, some materials used as an indicator may return to the pre-treatment state in color when the temperature is returned to normal temperature after the treatment, but with the heating identification system 100, since beet red B maintains the change at the maximum attained temperature even when returned to normal temperature after the high-temperature and high-pressure treatment, the beet red B can be expected to be applied as a temperature monitoring method and a process control.

Third, the heating identification system 100 eliminates the need for solvent substitution after nucleic acid extraction. That is, when the cell contents after extraction are used in subsequent steps, solvent substitution is unnecessary since there is almost no influence on the extract itself and the PCR enzyme, and the heating identification system 100 can be expected to be applied as a process control for the high-temperature and high-pressure method.

Fourth, the heating identification system 100 is highly safe because beet red B is a natural dye. That is, in the heating identification system 100, beet red B is a natural dye and a safe material that is recognized as a food additive and can be used in food factories or the like.

### 2. Details of Steps of Heating Identification System 100

Details of the steps of the heating identification system 100, which is a state identification method for identifying the temperature at which the sample has been heated, illustrated in FIG. 1 will be described. Hereinafter, the steps according to the embodiment will be described in the order of a bacterial culture step, a bacterial culture solution collection step, a dye addition step, a heating step, a dye confirmation step, a nucleic acid purification step, and a nucleic acid amplification step.

### 2-1. Bacterial Culture Step

Hereinafter, the bacterial culture step of culturing the bacteria C as a sample, which is performed prior to the bacterial culture solution collection step of the heating identification system 100, will be described.

### 2-1-1. Specific Example of Bacterial Culture Step

For example, in the bacterial culture step, the bacteria C is cultured in the bacterial culture solution S stored in the bacterial culture container 10. Describing an example of the instrument used for culture, in the bacterial culture step, a sterilized glass-stoppered conical flask is used as the bacterial culture container 10 to culture the bacteria C. Describing an example of the bacteria C to be cultured, E. coli and S. aureus are cultured in the bacterial culture step. Describing an example of culture conditions, in the bacterial culture step, the bacteria C is cultured overnight at 37° C. in an SCD liquid medium.

### 2-1-2. Method for Culturing Bacterial Culture Solution S

The bacterial culture solution S used in the bacterial culture step is obtained by culturing a sample containing a nucleic acid. The method for culturing a sample is not particularly limited, and examples thereof include a method in which a filter on which a sample has been collected is directly placed on a solid medium and the sample is cultured through the filter (solid phase culture). Another method for culturing a sample is, for example, a method in which a sample is cultured in the presence of a liquid medium or a solution obtained by dissolving a solid medium in water (liquid phase culture). In addition, the type of liquid medium or solid medium to be used is selected according to the type of sample to be cultured and physiological conditions.

### 2-1-3. Sample of Bacterial Culture Solution S

In the bacterial culture step, the sample to be treated is not particularly limited. For example, the sample to be treated may be a microorganism, animal cells other than microorganisms (for example, insect cells and the like) plant cells, a mycoplasma, a virus, or the like.

Examples of the microorganism include at least one species selected from the group consisting of Acinetobacter, Actinomyces, Aerococcus, Aeromonas, Alcaligenes, Bacillus, Bacteriodes, Bordetella, Branhamella, Brevibacterium, Campylobacter, Candida, Capnocytophagia, Chromobacterium, Clostridium, Corynebacterium, Cryptococcus, Deinococcus, Enterococcus, Erysipelothrix, Escherichia, Flavobacterium, Gemella, Haemophilus, Klebsiella, Lactobacillus, Lactococcus, Legionella., Leuconostoc, Listeria, Micrococcus, Mycobacterium, Neisseria, Cryptosporidium, Nocardia, Oerskovia, Paracoccus, Pediococcus, Peptostreptococcus, Propionibacterium, Proteus, Pseudomonas, Rahnella, Rhodococcus, Rhodospirillum, Staphylococcus, Streptomyces, Streptococcus, Vibrio, Yersinia, Methylobacterium, Ralstonia, and Sphingomonas species.

Some of the microorganisms described above take the form of spores depending on the growth state. In the heating identification system 100, the form of the sample to be treated is not particularly limited. In addition, in the heating identification system 100, the number of types of samples to be treated may be one or two or more.

### 2-2. Bacterial Culture Solution Collection Step

Hereinafter, the bacterial culture solution collection step of collecting the bacterial culture solution S, which is a sample solution, performed after the bacterial culture solution collection step of the heating identification system 100 will be described.

### 2-2-1. Specific Example of Bacterial Culture Solution Collection Step

For example, in the bacterial culture solution collection step, a part of the bacterial culture solution S stored in the bacterial culture container 10 is collected in the sealing container 20. Describing an example of the instrument used for collection, in the bacterial culture solution collection step, a sterilized glass hole pipette is used as a collecting instrument, and a sterilized glass tube with a stopper is used as the sealing container 20 to collect the bacterial culture solution S.

### 2-2-2. Sealing Container 20

In the bacterial culture solution collection step, the sealing container 20 is not particularly limited. For example, the sealing container 20 may be a stoppered glass tube, as well as a stoppered plastic tube, a microtube, or the like. In addition, it is sufficient that the sealing container 20 has a sealable structure and has durability against a temperature of about 140° C. in the heating step to be described later.

### 2-2-3. Others

In the bacterial culture solution collection step, the bacterial culture solution S may be pretreated before being collected in the sealing container 20. For example, the bacterial culture solution S may be a suspension containing the bacteria C after adding an enzyme and incubating the cells for a certain period of time. In addition, the bacterial culture solution S may be a suspension containing the bacteria C after centrifugation by a centrifuge to remove the medium components.

### 2-3. Dye Addition Step

Hereinafter, the dye addition step of adding a predetermined dye to the bacterial culture solution S containing the bacteria C as a sample, which is performed after the bacterial culture solution collection step of the heating identification system 100, will be described.

### 2-3-1. Specific Example of Dye Addition Step

For example, in the dye addition step, an aqueous solution obtained by dissolving beet red B, which is a red dye, in deionized water (DIW) is added. Here, in the dye addition step, the beet red B contains at least one of betanin and isobetanin. In the dye addition step, the dye to be added is not particularly limited as long as it has a visually recognizable color and the color disappears irreversibly in the heating temperature range of about 100° C. to 160° C.

### 2-3-2. Beet Red B

Beet red B will be described. Hereinafter, the properties of beet red B, the chemical structure of beet red B, and the natural dye containing beet red B will be described in this order.

### 2-3-2-1. Properties of Beet Red B

First, the properties of beet red B will be described. Beet red B is obtained by squeezing the red root of Beta vulgaris LINNE or extracting it with water, an acidic aqueous solution, or hydrous ethanol at room temperature to low temperature, and its main colors are betaine-based betanin and isobetanin. Beet red B is also known as a natural dye that is allowed as a food additive. Beet red B is a clear red dye as a property, has little color tone change due to pH (pH 4 to 7), and is well soluble in water but insoluble in anhydrous ethanol and fats. In addition, beet red B is unstable and fades with heat, and is also discolored by light or metal ions.

### 2-3-2-2. Chemical Structure of Beet Red B

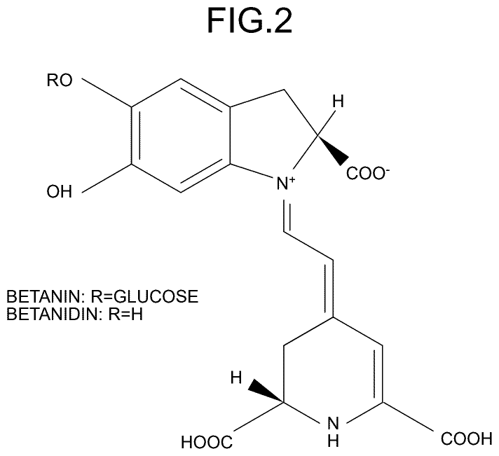

Second, the chemical structure of beet red B will be described with reference to FIG. 2. FIG. 2 is a diagram illustrating an example of a dye component of beet red B according to the embodiment. Beet red B is mainly composed of betanin and isobetanin. In the structural formula of FIG. 2, the structure in which the side chain R is glucose is betanin. Isobetanin is a stereoisomer of betanin. Beet red B may contain dextrin or lactose.

### 2-3-2-3. Natural Dye

Thirdly, natural dyes that are recognized as food additives, in which beet red B is classified. In addition to beet red in which beet red B is classified, natural dyes recognized as food additives include red cabbage color, red radish color, annatto extract, sepia color, turmeric oleoresin curcumin, cacao color, carotenes, gardenia red, gardenia blue, gardenia yellow, chlorophyll, Kaoliang color, cochineal extract, saffron color, perilla color, Sandalwood red, spirulina color, onion color, tamarind color, butterfly pea color, paprika color, tomato color, Hibiscus color, grape peel color, Haematococcus algae color, Monascus color, carthamus red, carthamus yellow, berry color, marigold color, purple sweet potato color, purple corn color, purple yam color, vegetable carbon black, and the like.

### 2-4. Heating Step

Hereinafter, a heating step of heating the sample solution, to which the predetermined dye has been added, and extracting nucleic acids from the cells of the bacteria C as the sample, which is performed after the dye addition step of the heating identification system 100, will be described.

### 2-4-1. Specific Example of Heating Step

For example, in the heating step, a solubilizing agent that promotes the dissolution of cells is added to the bacteria C as a sample in the sealing container 20 storing the heating solution H to which beet red B has been added in the dye addition step, the sealing container 20 is sealed by closing the lid, and the sealed sealing container 20 is heated at 140° C. for 45 seconds using the heating device 30 such as a heat block to extract nucleic acids from the cells of the bacteria C.

### 2-4-2. Type of Solubilizing Agent

In the heating step of the heating identification system 100, although the above effect can be achieved with water alone, it is preferable to add at least one solubilizing agent selected from the group consisting of a surfactant, an alkali, an acid, a redox agent, and a protein denaturant in addition to water for the purpose of more efficiently extracting nucleic acids from the sample. The solubilizing agent has the ability to dissolve the membrane structure of the sample. When the solubilizing agent acts on the membrane structure of the sample, the sample is easily broken, and nucleic acids can be more efficiently extracted from the sample. Hereinafter, types of the solubilizing agent will be described.

### 2-4-2-1. Surfactant

The surfactant used as a solubilizing agent may be, for example, ionic or nonionic. Examples of the nonionic surfactant include octylphenol ethoxylate (C₁₄H₂₂O(C₂H₄O)ₙ). In the nucleic acid extraction step of the heating identification system 100, commercially available octylphenol ethoxylate can be used, and examples thereof include Triton X-100 (C₁₄H₂₂O(C₂H₄O)ₙ, n=100) manufactured by SIGMA.

The ionic surfactant may be anionic, cationic, or amphoteric. Examples of the anionic surfactant include sodium dodecyl sulfate (SDS). Examples of the cationic surfactant include cetyltrimethylammonium bromide (CTAB). Examples of the amphoteric surfactant include betaine. Here, “betaine” is a generic term for compounds that have a positive charge and a negative charge at positions not adjacent to each other in the Same molecule, have no dissociable hydrogen atoms bonded to the atom having a positive charge, and have no charge as a whole molecule. Typical examples of betaine include trimethylglycine.

### 2-4-2-2. Alkali

Examples of the alkali used as the solubilizing agent include sodium hydroxide (NaOH) and potassium hydroxide (KOH).

### 2-4-2-3. Acid

Examples of the acid used as a solubilizing agent include hydrochloric acid (HCl) and sulfuric acid (H₂SO₄).

### 2-4-2-4. Redox Agent

Examples of the redox agent used as a solubilizing agent include hydrogen peroxide water, beta-mercaptoethanol, and dithiothreitol.

### 2-4-2-5. Protein Denaturant

Examples of the protein denaturant used as a solubilizing agent include guanidine hydrochloride and urea.

### 2-4-2-6. Others

As a component of the solubilizing agent, a chelating agent may be used. Examples of the chelating agent used as a solubilizing agent include ethylenediaminetetraacetic acid (EDTA).

In addition, among the above-described solubilizing agents, the solubilizing agent for the heating identification system 100 preferably contains a surfactant, and more preferably contains one or both of SDS and octylphenol ethoxylate.

For example, when it is desired to detect the nucleic acid extracted in the nucleic acid extraction step of the heating identification system 100 with high sensitivity, SDS is preferred. On the other hand, when the nucleic acids extracted in the nucleic acid extraction step of the heating identification system 100 are used for the enzyme reaction inhibited by SDS, octylphenol ethoxylate that acts milder on the membrane structure of the sample than SDS is preferably used.

The solubilizing agent of the heating identification system 100 may optionally include a buffer. Examples of the buffer include tris (hydroxymethyl) aminomethane hydrochloride (Tris-HCl).

### 2-4-3. Type of Nucleic Acid

In the heating step, the types of nucleic acids to be extracted is not particularly limited. For example, the nucleic acids to be extracted may be deoxyribonucleic acid (DNA) such as genomic DNA or plasmid DNA, or ribonucleic acid (RNA) such as messenger RNA, transfer RNA, or ribosomal RNA.

### 2-5. Dye Confirmation Step

Hereinafter, the dye confirmation step, which is an identification step performed after the heating step of the heating identification system 100 to identify the temperature at which the sample solution has been heated by the heating step based on the color tone change of a predetermined dye, will be described.

### 2-5-1. Specific Example of Dye Confirmation Step

For example, in the dye confirmation step, the maximum temperature at which the sample solution has been heated is identified to be 140° C. or higher by disappearance of the red color of beet red B. That is, in the dye confirmation step, when the red color of beet red B is visually disappeared and changed to yellow, it can be confirmed that the heating solution H containing the bacteria C has been heated to 140° C. or higher.

In the dye confirmation step, the maximum temperature at which the sample solution has been heated in the range of 100° C. to 160° C. is identified by measuring the red absorbance of beet red B. That is, in the dye confirmation step, the absorbance at 535 nm is measured using an absorptiometer, and it can be confirmed that the maximum temperature has reached 100° C. when the absorbance is 0.11, the maximum temperature has reached 110° C. when the absorbance is 0.10, the maximum temperature has reached 120° C. when the absorbance is 0.09, the maximum temperature has reached 130° C. when the absorbance is 0.07, the maximum temperature has reached 140° C. when the absorbance is 0.05, the maximum temperature has reached 150° C. when the absorbance is 0.04, and the maximum temperature has reached 160° C. when the absorbance is 0.02.

### 2-6. Nucleic Acid Purification Step

Hereinafter, the nucleic acid purification step, which is performed after the heating step of the heating identification system 100 to purify the nucleic acids extracted from the cells of the bacteria C, will be described.

### 2-6-1. Specific Example of Nucleic Acid Purification Step

For example, in the nucleic acid purification step, nucleic acids are purified by injecting the nucleic acids into a column having an adsorption carrier that adsorbs nucleic acids, and then injecting an eluate that elutes the nucleic acids into the column.

### 2-7. Nucleic Acid Amplification Step

Hereinafter, the nucleic acid amplification step, which is performed after the heating step or after the nucleic acid purification step of the heating identification system 100 to amplify the nucleic acids extracted from the cells of the bacteria C, will be described.

### 2-7-1. Specific Example of Nucleic Acid Amplification Step

For example, in the nucleic acid amplification step, the nucleic acids are amplified by performing PCR on the solution containing the nucleic acids extracted in the heating step. In the nucleic acid amplification step, nucleic acids are amplified by performing PCR on the solution containing the nucleic acids purified in the nucleic acid purification step.

### 3. Results of Various Experiments

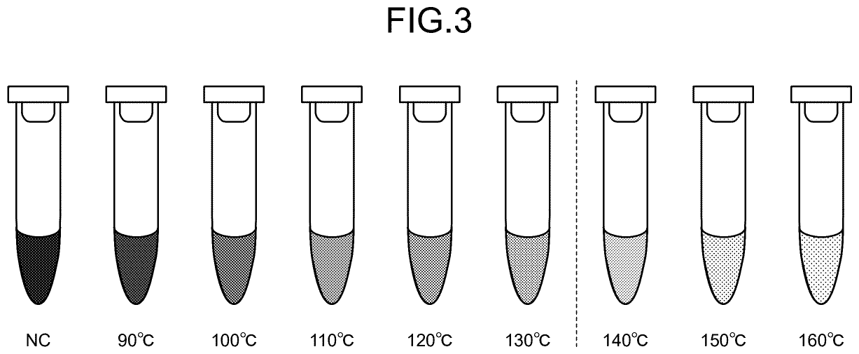

Various experimental results by the heating identification system 100 according to the embodiment will be described with reference to FIGS. 3 to 10. Hereinafter, the experimental results related to the heating confirmation experiment and the experimental results related to the PCR amplicon measurement experiment will be described in this order.

### 3-1. Heating Confirmation Experiment

Experimental results 1 to 3 related to the heating confirmation experiment for confirming the heating temperature by the heating identification system 100 will be described with reference to FIGS. 3 to 5. FIGS. 3 to 5 are diagrams illustrating examples of experimental results of the heating confirmation experiment according to the embodiment. Hereinafter, the experimental results 1 to 3 of the heating confirmation experiment will be sequentially described while illustrating the experimental procedure.

### 3-1-1. Experimental Procedure of Heating Confirmation Experiment

An example of an experimental procedure regarding a heating confirmation experiment by the heating identification system 100 will be described. First, as beet red B, a beet red solution is prepared by dissolving a powder “Sun Beet LF” manufactured by San-Ei Gen F. F. I., Inc. in deionized water at a concentration of 0.05 g/mL. Second, 2 microliters of the prepared beet red solution is added to 38 microliters of a solubilizing agent solution containing 1% SDS and Tris-HCl. Thirdly, 40 microliters of the mixed solution of beet red B and a solubilizing agent is sealed in a heat-resistant microtube and heated for 45 seconds. Fourth, the absorbance is measured with an absorptiometer “NanoDrop (UV-VIS)” manufactured by Thermo Fisher Scientific.

### 3-1-2. Experimental Result 1 of Heating Confirmation Experiment

Experimental result 1 of the heating confirmation experiment will be described with reference to FIG. 3. FIG. 3 illustrates respective states of the microtubes: negative control sample “NC” which is an unheated mixed solution, “90° C.” which is a mixed solution heated at 90° C., “100° C.” which is a mixed solution heated at 100° C., “110° C.” which is a mixed solution heated at 110° C., “120° C.” which is a mixed solution heated at 120° C., “130° C.” which is a mixed solution heated at 130° C., “140° C.” which is a mixed solution heated at 140° C., “150° C.” which is a mixed solution heated at 150° C., and “160° C.” which is a mixed solution heated at 160° C.

### 3-1-3. Consideration of Experimental Result 1 of Heating Confirmation Experiment

In FIG. 3, no disappearance of red color is observed in “NC”, but it can be visually recognized that the degree of disappearance of red color increases as the heating temperature increases. In FIG. 3, the degree of disappearance of the red color is particularly remarkable after 140° C. indicated by a broken line.

From the experimental result 1 of the heating confirmation experiment, in the heating identification system 100, it is possible to confirm that each sample has been heated to 140° C. or higher by visually recognizing the color tone change of the beet red B by heating at 140° C. for 45 seconds, which is the treatment condition of the high-temperature and high-pressure method described above.

### 3-1-4. Experimental Result 2 of Heating Confirmation Experiment

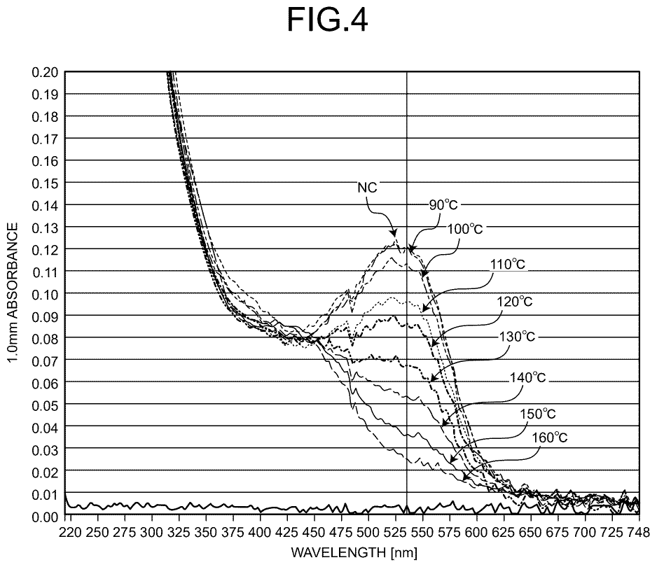

Experimental result 2 of the heating confirmation experiment will be described with reference to FIG. 4. FIG. 4 illustrates respective absorption spectra of the mixed solutions: negative control sample “NC” which is an unheated mixed solution, “90° C.” which is a mixed solution heated at 90° C., “100° C.” which is a mixed solution heated at 100° C., “110° C.” which is a mixed solution heated at 110° C., “120° C.” which is a mixed solution heated at 120° C., “130° C.” which is a mixed solution heated at 130° C., “140° C.” which is a mixed solution heated at 140° C., “150° C.” which is a mixed solution heated at 150° C., and “160° C.” which is a mixed solution heated at 160° C.

### 3-1-5. Experimental Result 3 of Heating Confirmation Experiment

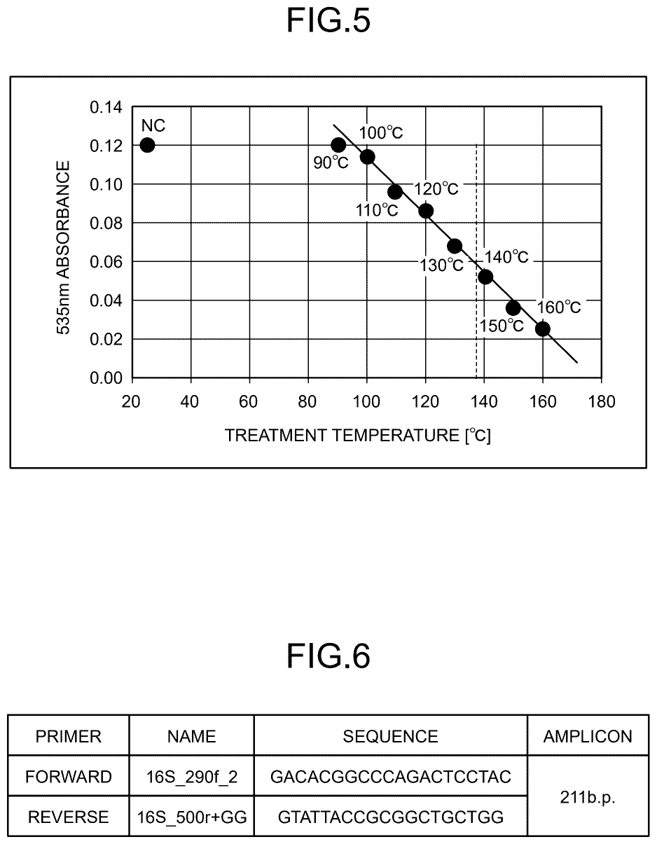

Experimental result 3 of the heating confirmation experiment will be described with reference to FIG. 5. FIG. 5 illustrates respective absorbances of the mixed solutions at a wavelength of 535 nm: negative control sample “NC” which is an unheated mixed solution, “90° C.” which is a mixed solution heated at 90° C., “100° C.” which is a mixed solution heated at 100° C., “110° C.” which is a mixed solution heated at 110° C., “120° C.” which is a mixed solution heated at 120° C., “130° C.” which is a mixed solution heated at 130° C., “140° C.” which is a mixed solution heated at 140° C., “150° C.” which is a mixed solution heated at 150° C., and “160° C.” which is a mixed solution heated at 160° C.

### 3-1-6. Consideration of Experimental Results 2 and 3 of Heating Confirmation Experiment

In FIG. 4, it can be confirmed that the absorbance decreases in the order of “NC”, “90° C.”, “100° C.”, “110° C.”, “120° C.”, “130° C.”, “140° C.”, “150° C.”, and “160° C.”. In FIG. 5, it can be confirmed that the absorbance decreases linearly in the order of “NC”, “90° C.”, “100° C.”, “110° C.”, “120° C.”, “130° C.”, “140° C.”, “150° C.”, and “160° C.” with respect to the absorbance measured at a wavelength of 535 nm indicated by the solid line in FIG. 4.

From the experimental results 2 and 3 of the heating confirmation experiment, in the heating identification system 100, the maximum attained temperature of each sample can be identified by measuring the absorbance of the beet red B after the heating treatment.

### 3-2. PCR Amplicon Measurement Experiment

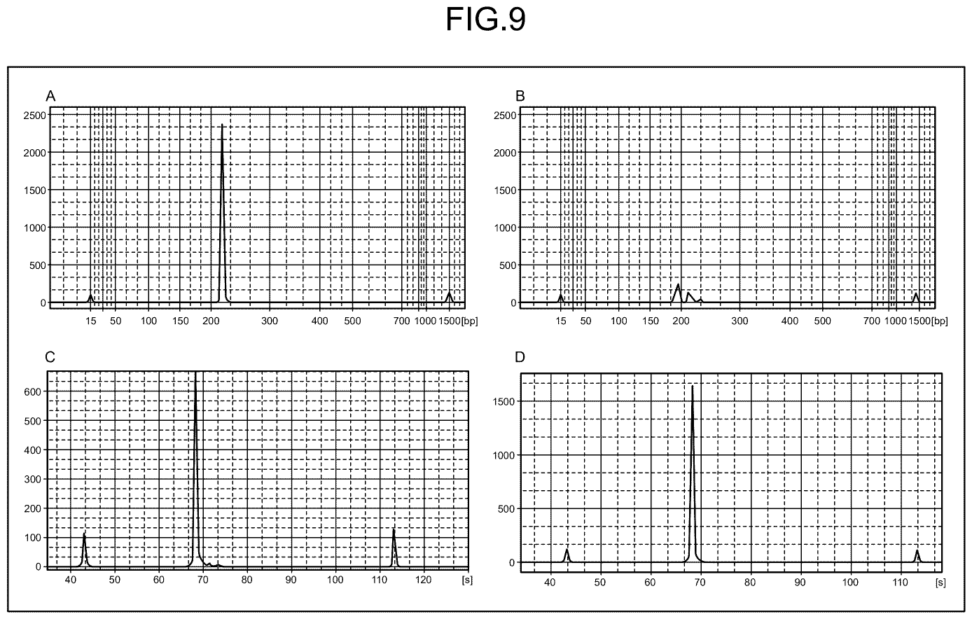

Experimental results 1 and 2 relating to the PCR amplicon measurement experiment for measuring the PCR amplicon by the heating identification system 100 will be described with reference to FIGS. 6 to 10. FIGS. 6 to 8 are diagrams illustrating examples of experimental conditions of the PCR amplicon measurement experiment according to the embodiment. FIGS. 9 and 10 are diagrams illustrating examples of the experimental results of the PCR amplicon measurement experiment according to the embodiment. Hereinafter, the experimental results 1 and 2 of the PCR amplicon measurement experiment will be sequentially described while showing the experimental procedures.

### 3-2-1. Experimental Procedure of PCR Amplicon Measurement Experiment

An example of the experimental procedure related to the PCR amplicon measurement experiment by the heating identification system 100 will be described with reference to FIGS. 6 to 8. First, beet red B is added to a bacterial suspension obtained by culturing E. coli NBRC 3972 strain overnight in an SCD medium, and a high-temperature and high-pressure treatment (heating at 140° C. for 45 seconds) by the high-temperature and high-pressure method is performed. Second, the mixed solution after the high-temperature and high-pressure treatment is diluted to 1/100 to prepare a diluted solution. Third, 20 microliters of the diluted solution and 20 microliters of the PCR mix shown in the experimental conditions 1 and 2 are mixed, and PCR is performed under the temperature cycle conditions shown in the experimental conditions 3. Fourth, the solution after PCR is subjected to electrophoresis using “Agilent 2100 Bioanalyzer electrophoresis system” manufactured by Agilent Technologies, and the presence or absence of PCR amplification is confirmed.

### 3-2-1-1. Experimental Conditions 1

First, the experimental conditions 1 for primers in the PCR amplicon measurement experiment will be described with reference to FIG. 6. As illustrated in the example of FIG. 6, the forward primer has the primer name “16S 290 f_2”, and the base sequence “GACACGGCCCAGACTCCTAC”. The reverse primer has the primer name “16S 500r+GG”, and the base sequence “GTATTACCGCGGCTGCTGG”. Further, the amplicon base pair size is “211 b.p.” .

### 3-2-1-2. Experimental Conditions 2

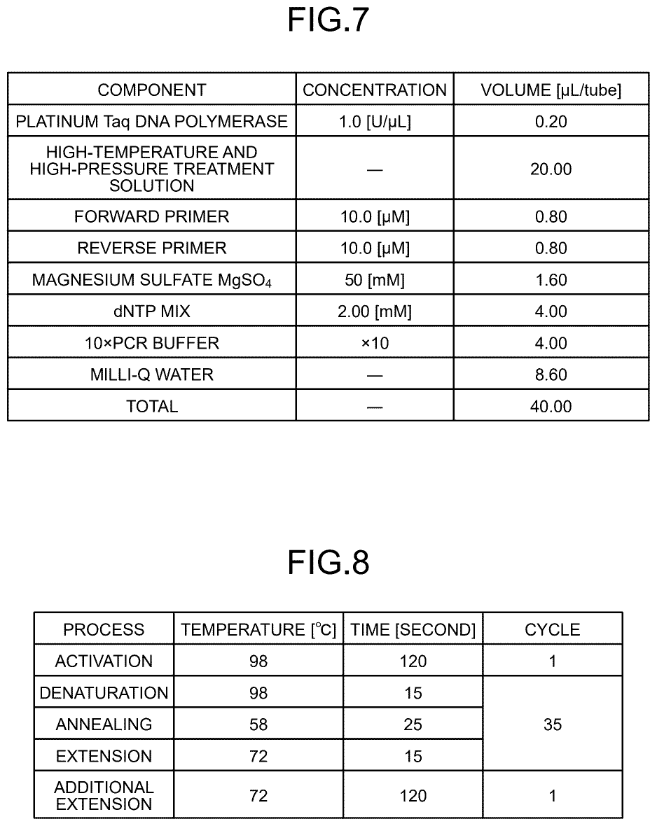

Second, the experimental conditions 2 for the reagent of the PCR amplicon measurement experiment will be described with reference to FIG. 7. As illustrated in the example of FIG. 7, as reagents for the PCR amplicon measurement experiment, 1.0 U/microliter and 0.20 microliter/tube of “Platinum Taq DNA polymerase”, 20 microliters/tube of “high-temperature and high-pressure treatment solution”, 10.0 micromolars and 0.8 microliter/tube of “forward primer”, 10.0 micromolars and 0.8 microliter/tube of “reverse primer”, 50 mM and 1.60 microliters/tube of “magnesium sulfate MgSO4”, 2.00 mM and 4.00 microliters/tube of “deoxynucleoside triphosphate (dNTP) mix”, 4.00 microliters/tube of “10 X PCR buffer” at 10 fold concentration, and 8.60 microliters/tube of “Milli-Q water” are mixed to make a total of 40.00 microliters/tube.

### 3-2-1-3. Experimental Conditions 3

Third, the experimental conditions 3 regarding the reaction conditions of the PCR amplicon measurement experiment will be described using FIG. 8. As illustrated in the example of FIG. 8, as reaction conditions for the PCR amplicon measurement experiment, the “Activation” process is performed at 98° C. for 120 seconds, for one cycle. A DNA elongation reaction consists of three steps: “Denaturation”, “Annealing”, and “Extension”. The “Denaturation” process is performed at 98° C. for 15 seconds, the “Annealing” process is performed at 58° C. for 25 seconds, and the “Extension” process is performed at 72° C. for 15 seconds. These three steps are performed as one cycle, and 35 cycles are performed. In addition, the “Additional Extension” process is performed at 72° C. for 120 seconds, for one cycle.

### 3-2-2. Experimental Result 1 of PCR Amplicon Measurement Experiment

Experimental result 1 of the PCR amplicon measurement experiment will be described with reference to FIG. 9. FIG. 9A is an electrophoretic diagram of a positive control sample “PC”, which is a mixed solution without beet red B. FIG. 9B is an electrophoretic diagram of a negative control sample “NC”, which is a mixed solution not subjected to high-temperature and high-pressure treatment. FIG. 9C is an electrophoretic diagram of the “beet red method (1/100 dilution, with a solubilizing agent)”, which is a mixed solution with beet red B and a solubilizing agent. FIG. 9D is an electrophoretic diagram of the “beet red method (without a solubilizing agent)”, which is a mixed solution with beet red B and no solubilizing agent.

### 3-2-3. Experimental Result 2 of PCR Amplicon Measurement Experiment

Experimental result 2 of the PCR amplicon measurement experiment will be described with reference to FIG. 10. In the positive control sample “PC”, which is a mixed solution without beet red B illustrated in FIG. 10A, the molar concentration of the PCR amplicon is 307.2 nmol/L. In the negative control sample “NC”, which is a mixed solution not subjected to the high-temperature and high-pressure treatment illustrated in FIG. 10B, the molar concentration of the PCR amplicon is 38.8 nmol/L. In the “beet red method (1/100 dilution, with a solubilizing agent)”, which is a mixed solution with beet red B and a solubilizing agent illustrated in FIG. 10C, the molar concentration of the PCR amplicon is 112.9 nmol/L. In the “beet red method (without solubilizing agent)”, which is a mixed solution with beet red B and no solubilizing agent illustrated in FIG. 10D, the molar concentration of the PCR amplicon is 255.0 nmol/L.

### 3-2-4. Consideration of Experimental Results 1 and 2 of PCR Amplicon Measurement Experiment

From FIGS. 9 and 10, the results of PCR performed using the mixed solutions with beet red B (FIGS. 9D and 10D) are 83% with respect to the positive control sample “PC”, which is the result of PCR using the mixed solution without beet red B (FIGS. 9A and 10A).

From the experimental results 1 and 2 of the PCR amplicon measurement experiment, it can be confirmed that the heating identification system 100 does not affect the nucleic acid extraction step and the nucleic acid amplification step.

### 4. Treatment Flow of Heating Identification System 100

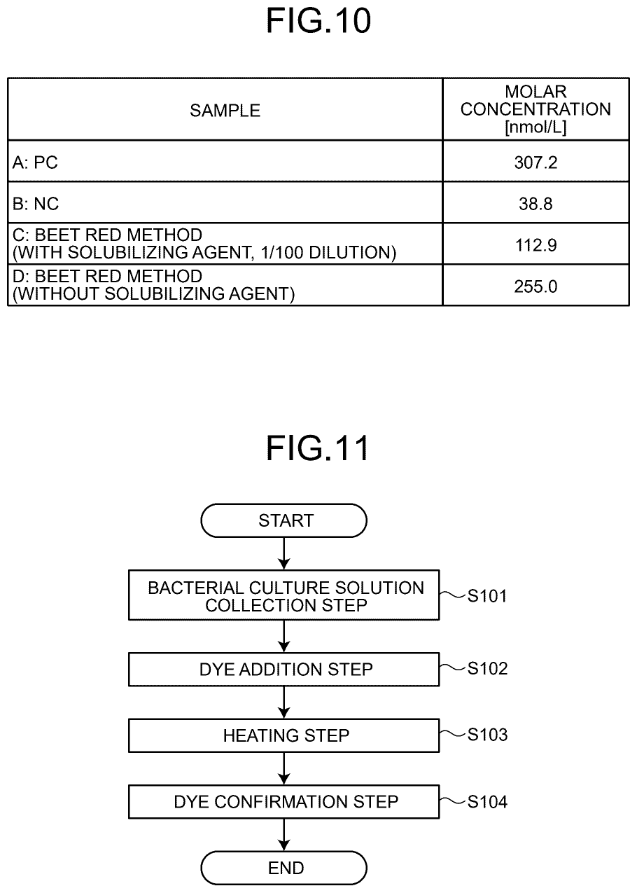

The flow of steps of the heating identification system 100 according to the embodiment will be described with reference to FIG. 11. FIG. 11 is a flowchart illustrating an example of the flow of the heating identification step according to the embodiment. Note that the following steps S101 to S104 may be performed in a different order. In addition, some of the following steps S101 to S104 may be omitted.

First, in the heating identification system 100, the bacterial culture solution collection step (step S101) is performed. Second, in the heating identification system 100, the dye addition step (step S102) is performed. Third, in the heating identification system 100, the heating step (step S103) is performed. Fourth, in the heating identification system 100, the dye confirmation step is performed (step S104), and the heating identification step is completed. Note that in the heating identification system 100, the nucleic acid purification step and the nucleic acid amplification step may be performed after performing the dye confirmation step.

5. Effects of embodiment Finally, effects of the embodiment will be described.

Hereinafter, the effects 1 to 8 corresponding to the steps according to the embodiment will be described.

### 5-1. Effect 1

First, in the steps according to the above-described embodiment, a predetermined dye is added to a sample solution containing a sample, the sample solution with the predetermined dye is heated, and the heating temperature at which the sample solution has been heated is identified based on the color tone change of the predetermined dye. Therefore, in the step according to the embodiment, the heating temperature of the sample can be effectively identified.

### 5-2. Effect 2

Second, in the step according to the above-described embodiment, the predetermined dye is beet red B, which is a red dye. Therefore, in the step according to the embodiment, the heating temperature of the sample can be effectively identified by using a highly visible and highly safe dye.

### 5-3. Effect 3

Third, in the step according to the above-described embodiment, beet red B contains at least one of betanin and isobetanin. Therefore, in the step according to the embodiment, the heating temperature of the sample can be effectively identified by using a dye whose color tone reversibly changes according to the heating temperature.

### 5-4. Effect 4

Fourth, in the step according to the above-described embodiment, the maximum temperature at which the sample solution has been heated is identified to be 140° C. or higher by the disappearance of the red color of beet red B. Therefore, in the step according to the embodiment, the heating temperature of the sample can be effectively identified in the heating treatment at 140° C. or higher.

### 5-5. Effect 5

Fifth, in the step according to the above-described embodiment, the maximum temperature at which the sample solution has been heated in the range of 100° C. to 160° C. is identified by measuring the absorbance of the red color of beet red B. Therefore, in the step according to the embodiment, the heating temperature of the sample can be effectively identified in the heating treatment in the range of 100° C. to 160° C.

### 5-6. Effect 6

Sixth, in the step according to the above-described embodiment, the sample solution is the bacterial culture solution S containing the bacteria C.

Therefore, in the step according to the embodiment, the heating temperature of the sample can be effectively identified in the high-temperature and high-pressure method for extracting nucleic acids from the cells of the bacteria C.

### 5-7. Effect 7

Seventh, in the step according to the above-described embodiment, the nucleic acids extracted from the cells of the bacteria C is purified. Therefore, in the step according to the embodiment, the heating temperature of the sample can be effectively identified without affecting the purification of the nucleic acids extracted from the cells of the bacteria C.

### 5-8. Effect 8

Eighth, in the step according to the above-described embodiment, the nucleic acids extracted from the cells of the bacteria C is amplified. Therefore, in the step according to the embodiment, the heating temperature of the sample can be effectively identified without affecting the amplification of the nucleic acids extracted from the cells of the bacteria C.

### System

The treatment procedure, the control procedure, the specific name, and the information including various data and parameters illustrated in the above text and drawings can be changed as desired unless otherwise specified.

In addition, each component of each device illustrated in the drawings is a functional concept, and is not necessarily physically configured as illustrated in the drawings. That is, specific forms of distribution and integration of the devices are not limited to those illustrated in the drawings. That is, all or a part thereof can be functionally or physically distributed and integrated in arbitrary units according to various loads, usage conditions, and the like.

### REFERENCE SIGNS LIST

10 Bacterial culture container
20 Sealing container
30 Heating device
100 Heating identification system

1. A state identification method for identifying the temperature at which a sample has been heated, comprising:
adding a predetermined dye to a sample solution including the sample;
heating the sample solution to which the predetermined dye has been added; and
identifying the temperature at which the sample solution has been heated step-based on the color tone change of the predetermined dye.

2. The state identification method according to claim 1, wherein
the predetermined dye is beet red, which is a red dye.

3. The state identification method according to claim 2, wherein
the beet red includes at least one of betanin and isobetanin.

4. The state identification method according to claim 2, wherein
the maximum temperature at which the sample solution has been heated is identified to be 140° C. or higher by the disappearance of the red color of the beet red

5. The state identification method according to claim 2, wherein
the maximum temperature at which the sample solution has been heated in the range of 100° C. to 160° C. is identified by measuring the red absorbance of the beet red.

6. The state identification method according to claim 1 wherein
the sample solution is a bacterial culture solution including bacteria.

7. The state identification method according to claim 6, further comprising
purifying the nucleic acids extracted from the cells of the bacteria.

8. The state identification method according to claim 6, further comprising
amplifying the nucleic acids extracted from the cells of the bacteria.
